(19) United States
(12) Patent Application Publication
(10) Pub. No.: US 2021/0154270 A1
(43) Pub. Date: May 27, 2021

(54) ANTI-AGING COMPOSITIONS AND METHODS OF USE

(71) Applicant: YISSUM RESEARCH DEVELOPMENT COMPANY OF THE HEBREW UNIVERSITY OF JERUSALEM LTD., Jerusalem (IL)

(72) Inventors: Miriam GRUNEWALD, Elazar (IL); Eli KESHET, Aminadav (IL)

(21) Appl. No.: 17/046,807

(22) PCT Filed: Apr. 11, 2019
(86) PCT No.: PCT/IL2019/050409

§ 371 Date: Oct. 12, 2020

Related U.S. Application Data
(60) Provisional application No. 62/656,471, filed on Apr. 12, 2018.

(51) Int. Cl. A61K 38/18; A61P 25/28

(52) U.S. Cl. CPC A61K 38/1866; A61P 25/28

(57) ABSTRACT

The present invention is directed to methods and compositions for treating or attenuating age-related symptoms or diseases in a cell, a tissue, an organ, or an organism by administration of therapeutically effective amounts of VEGF stimulating-, VEGFR stimulating-compound, or any combination thereof.

### CROSS-REFERENCE TO RELATED APPLICATIONS

This application claims the benefit of priority of U.S. Provisional Patent Application No. 62/656,471 titled “ANTI-AGING COMPOSITIONS AND METHODS OF USE”, filed Apr. 12, 2018, the contents of which is incorporated herein by reference in its entirety.

### FIELD OF INVENTION

The present invention is in the field of anti-aging pharmacology

### BACKGROUND OF INVENTION

Aging can be defined as an inevitable, irreversible decline in organ function that occurs over time even in the absence of injury, illness, environmental risks, or poor lifestyle choices (e.g., unhealthy diet, lack of exercise, substance abuse). Initially, the changes in organ function do not affect baseline function; the first manifestations are a reduced capacity of each organ to maintain homeostasis under stress (e.g., illness, injury). The cardiovascular, renal, and central nervous systems are usually the most vulnerable.

Various diseases interact with pure aging effects to cause geriatric-specific complications, particularly in the cardiovascular, renal, and central nervous systems, even when those organs are not the primary ones affected by a disease. Typical examples are delirium complicating pneumonia or urinary tract infection (UTI) and the falls, dizziness, syncope, urinary incontinence, and weight loss that often accompany many minor illnesses in the elderly. Aging organs are also more susceptible to injury; e.g., intracranial hemorrhage is more common and is triggered by less clinically important injury in the elderly.

As the average life span increases in developed countries, which is primarily attributed to medical breakthroughs and improvements in nutrition and lifestyle, treatments that can slow aging or treat aging-related disorders are greatly in need.

### SUMMARY OF THE INVENTION

The present invention, in some embodiments thereof, is directed to compositions and methods for attenuating age-related diseases using vascular endothelial growth factor (VEGF) signaling stimulating compounds.

According to one aspect there is provided a method for preventing or treating an age-related disorder or symptoms thereof in a subject, the method comprising administering to the subject a pharmaceutical composition comprising a therapeutically effective amount of vascular endothelial growth factor (VEGF)-stimulating compound and an acceptable carrier, wherein the composition constantly maintains VEGF plasma levels in the subject by at most 3-fold compared to a baseline, thereby preventing or treating an age-related disorder or symptoms thereof in the subject.

According to another aspect, there is provided a method for extending the lifespan of a cell, tissue, an organ, or an organism, the method comprising the step of constantly maintaining VEGF levels in the cell, the tissue, the organ, or the organism by at most 3-fold compared to a baseline, thereby extending the lifespan of the cell, the tissue, the organ, or the organism.

According to another aspect, there is provided a pharmaceutical composition comprising VEGF-stimulating compound in an amount effective to increase VEGF signaling by 3-fold at most in a subject's plasma compared to a baseline.

In some embodiments, the VEGF plasma levels comprise free VEGF plasma levels.

In some embodiments, the administering is for at least 30 days before appearance of the age-related disorder or symptom thereof.

In some embodiments, constantly is for at least 30 days.

In some embodiments, the subject is afflicted with chronic ischemia.

In some embodiments, the subject afflicted with chronic ischemia has plasma lactate levels of 2-5 mmol/L.

In some embodiments, the age-related disorder or symptom is selected from the group consisting of: muscle weakness, cold intolerance, skin wrinkles, reduced skin healing, weight loss, weight gain, cognitive impairment, kyphosis, reduced bone mineralization, inhibition or lack of brown adipose tissue activity, and subdermal fat loss.

In some embodiments, the age-related disorder is selected from the group consisting of: muscle wasting disease, osteoporosis, pancreatic disease, intestinal disease, neoplastic lesions, and hepatic disease.

In some embodiments, the VEGF-stimulating compound is selected from the group consisting of: a nucleic acid, a peptide, a polypeptide, a peptidomimetic, a carbohydrate, a lipid, a small organic molecule, and an inorganic molecule.

In some embodiments, the VEGF-stimulating compound is selected from the group consisting of: VEGF, VEGF Receptor (VEGFR)-stimulating compound, or any combination thereof.

In some embodiments, the baseline is VEGF basal levels in a tissue of the subject.

In some embodiments, the method comprises the step of administrating to the cell, the tissue, the organ, or the organism a pharmaceutical composition comprising a therapeutically effective amount of a VEGF-stimulating compound.

In some embodiments, the administering is for at least 30 days.

In some embodiments, the composition further comprises a VEGFR-stimulating compound.

In some embodiments, VEGF-stimulating compound, VEGFR-stimulating compound, or both, is VEGF.

In some embodiments, the composition is for use in extending lifespan of a cell, a tissue, an organ, or an organism.

In some embodiments, extending lifespan is by preventing or treating an age-related symptom or disease in the cell, the tissue, the organ, or the organism.

In some embodiments, the organism is afflicted by chronic ischemia.

In some embodiments, the increased VEGF signaling is for at least 30 days.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a graph describing VEGF plasma levels in mice. Control individuals (Control) had basal low VEGF plasma levels, that were significantly lower (p<0.0005) compared to individuals over-expressing VEGF (VEGF), from the 8^{th} month onwards. A gradual increase in control individuals was observed from month 20 onwards. Measurements of VEGF levels in the plasma had ceased on month 26 in the control group due to 100% mortality or poor health conditions, while persisted for the VEGF over-expressing animals until month 36.

FIGS. 2A-2B are graphs describing survival proportion among males (2A) and females (2B) mice. Individuals over-expressing VEGF (VEGF) outlived control (Control). All control mice died by month 31.5 (males; 2A) and by month 30 (females; 2B), during which survival was 52% (males; 2A) and 59% (females; 2B) for VEGF mice. VEGF mice maximum lifespan was extended to 37.6 months (males; 2A) and 36.46 months (females; 2B). The median survival for control mice was 22.7 months (males; 2A) and 22.2 months (females; 2B), whereas it was 33 months for VEGF male mice and 30.85 months for VEGF female mice. Differences in the survival curves were found to be statistically significant with a P value lower than 0.0001 (Gehan-Breslow-Wilcoxon Test). A month was considered to have an average of 30.42 days.

FIGS. 3A-3C are graphs and images describing weight gain of male mice. (3A) Starting from 12 months of age, VEGF male mice gained significantly less weight than their control littermates. Body composition analysis by Echo-MRI showed less fat accumulation in VEGF mice at 16 months of age compared to their control littermates (3B). VEGF mice maintain their weight throughout their adult life while a significant weight loss was observed in aged control mice (3B). At day of sacrifice, control individuals were significantly more cachectic than their VEGF littermates, weighing 27±3 gr and 40±5 gr, respectively. (3C) overview image of 24 months old control and VEGF mice.

FIGS. 4A-4G are vertical bar graphs comparing various behavior and metabolic parameters between control (white bars) and VEGF (black bars) mice. VEGF mice were found to have significantly increased (females) or similar (males, not shown) food intake (4A), and similar water intake (4B). Both control and VEGF mice were found to have similar ambulatory (4C) and voluntary activities (4D). (4E-4F) are graphs describing the relative amount of fat (4E) and carbohydrate (4F) oxidation (based on measurements of oxygen consumption and CO₂ exhalation) demonstrating that VEGF mice conserved a higher metabolic flexibility while aging also reflected by FIG. 4G. (4G) is a graph showing the respiratory quotient (RQ). RQ was calculated from the ratio of carbon dioxide produced by the body of the mouse to oxygen consumed by the body, indicating which macronutrients were being metabolized (0.7 for solely fat, 0.8 for solely proteins, 1 for solely carbohydrates).

FIGS. 5A-5I are graphs and micrographs demonstrating comparative dynamics in white adipose tissue (WAT) weight and activity. At 18 months, a significant reduction in WAT mass was observed in VEGF mice compared to their control littermates (5A), accompanied by adequate perfusion maintenance of this tissue (as reflected by the number of endothelial cells per gram tissue; 5B). (5C-5E) are micrographs of histological sections of abdominal WAT isolated from control (5C) and VEGF-overexpressing mice (5D-5E). Specific staining for uncoupling protein 1 (UCP1) highlighted islands of beige adipocytes, which are known to have high thermogenic capacity, only in the VEGF mice (5D). (5E) is an enlarged area defined by a square in 5D. In addition, control WAT was found to accommodate larger immune cell infiltrates (5F-5G), compared to WAT obtained from VEGF (5H-5I). (5G) and (5I) are enlarged areas defined by squares in 5F and 5H, respectively. Scale bar: 5C-5H=200 μm; 5E=50 μm; 5G and 5I=20 μm.

FIG. 6 is a graph showing a better glucose tolerance in 18 months old VEGF male mice compared to their control littermates. AL—ad libitum.

FIGS. 7A-7H are pictures and graphs showing differential liver structurality and functionality for control and VEGF mice. At 24 months of age, livers of control mice presented advanced steatosis (7A) characterized by lipid accumulation (evidenced by Oil-red O staining as large gray droplets; 7C) as well hepatocytes' injury, as demonstrated by increased serum concentrations of ALT (7E) and AST (7F). In contrast, VEGF mice were found to be protected from aging-associated hepatic steatosis and hepatocytes injury (7C-7F). Such lipid accumulations were not observed in VEGF-overexpressing mice (7B and 7D). Bars=50 μm. (7G-7H) are high magnification electron microscopy images of hepatocytes from livers of control and VEGF mice. Mitochondria observed in control hepatocytes were enlarged and displayed disorganized cristae (7G). The rough endoplasmic reticulum (RER) appeared to be swollen and sparsely decorated with ribosomes which indicated ATP depletion induced-stress in control hepatocytes. In contrast, mitochondria and RER in VEGF mice were shown to have a normal appearance (7H). Scale bar=1,000 nm in 7G-7H.

FIGS. 8A-8D are micrographs of histological sections of dorsal skin tissues obtained from 24 months old control (8A-8B) and VEGF over-expressing (8C-8D) mice, which were stained with Masson's trichrome stain. A very few adipocytes were observed in sections of control mice skin (8A) reflecting a case of major hypodermal fat loss. In contrast, VEGF over-expressing mice, demonstrated a rich layer of adipose tissue (8B and 8D). 8C and 8D are enlargements of areas from 8A and 8B, respectively. Scale bar: (8A-8B)=200 μm; (8C-8D)=50 μm.

FIG. 9 is a graph describing performance of mice following a ROTAROD test that evaluates balance, grip strength and motor coordination and is measured as seconds before falling. At 15 months of age and onward, VEGF mice (▪) showed better performance than their control littermates (●).

FIG. 10A-10C are images and a graph demonstrating differential kyphosis index (KI) for control and VEGF mice. (10A-10B) are representative high resolution X-ray radiographs of control (10A) and VEGF (10B) 24 months old mice which were sedated (50 mg/kg ketamine and 10 mg/kg xylazine HCl administered by s.c.i.), lightly taped to the table support, radiographed (52 kVp, 4.30 mA) and had their KI calculated. At 24 months of age, the KI of control mice was found to be significantly lower than that of the VEGF-overexpressing mice (by approximately 1.5-fold; 10C). Further, VEGF mice were shown to maintain their KI when comparing it at the ages of 12 months and 24 months (10C). AB—connecting line drawn from posterior edge of C7 (A) to posterior edge of L6 (B); CD—connecting line drawn from dorsal border of vertebral body farthest from AB line; KI=AB/CD. Lower KI indicates more severe kyphosis.

FIGS. 11A-11D are high resolution micro computational tomography (CT) images of cross sections (see inset in 11C for section orientation) through femoral bones of control (11A-11B) and VEGF-overexpressing mice (11C-11D). Bone morphometry was assessed by measuring bone to tissue volume ratio. In average, control mice lose 22% more bone tissue than their VEGF littermates.

FIGS. 12A-12F are fluorescent images of bone tissue. Bones were dissected from control A, B and C) and VEGF over-expressing (VEGF; D, E and F) 15 months old mice, decalcified, sectioned and immunostained with the endothelial markers Endomucin (12A and 12D) and CD31 (12B and 12E). CD31⁺EMCN^{NEG} elongated (white arrow heads) and branched (black arrow heads) arteries were observed in the diaphysis of VEGF over-expressing mice (12F). Imaging was done using a confocal laser-scanning microscopy, using the z-stack scanning to obtain sequential depth imaging of thick bone sections. Three dimensional (3D) reconstructions of images were done using Imaris software. Bar=50 μm.

FIGS. 13A-13D are micrographs of the pancreas. Pancreatic tissues were dissected from control (13A-13B) and VEGF over-expressing (13C-13D) 24 months old mice, fixed in formalin and processed for paraffin embedding. Sections of 6 μm were stained with standard Hematoxylin and Eosin stain. In sections of control mice, a substantial pancreatic steatosis was observed in numerous lobes and lobules (13A-13B). In contrast, sections of VEGF over-expressing mice demonstrated hallmark features of a healthy pancreas, as demonstrated by packed lobes and lobules comprising both endocrine islets and exocrine acini (13C-13D). (13B) and (13D) represent enlargements of the regions defined in squares in (13A) and (13C), respectively.

FIGS. 14A-14D are images of histological sections of colon (14A and 14C) and duodenum (14B and 14D) tissues, from control (14A-14B) and VEGF over expressing (14C-14D) mice. Intestine tissues were dissected from 24 months old mice, fixed in formalin and processed for paraffin embedding. Sections of 6 μm were stained with standard Hematoxylin and Eosin stain. In sections of control mice colon, villi were found to be short and aged in appearance, starved, uniformly spaced and oddly shaped with large immune aggregates, undigested food particles and disordered villi were also observed (14A). Sections of the duodenum revealed fat deposition, extensive adenomas, undigested food particles and the presence of numerous early polyps (14B). In contrast, colon and duodenum villi in VEGF-overexpressing mice were completely normal in appearance with no documentation of adenoma. Nevertheless, some early polyps were observed (14C-14D).

FIGS. 15A-15B are graphs describing decrease in cancer-related phenomena in VEGF-overexpressing mice compared to control. (15A) is a graph showing the % of mice which presented at least one spontaneous tumor type at time of sacrifice. In both female and male control mice, neoplastic lesions were observed more often than in their VEGF littermates. (15B) is a graph showing a significant increase in circulating granulocytes in the blood of control mice as compared to their VEGF littermates.

FIG. 16 is a graph describing the increase in circulating soluble VEGF Receptor 1 (VEGFR1, i.e., sFlt1) in aging control mice. During the last months of life, control aged mice comprised significantly higher sFlt1levels compared to control young mice. Each group age comprised more than 8 mice.

### DETAILED DESCRIPTION OF THE INVENTION

The present invention, in some embodiments thereof, relates to VEGF signaling stimulating-compositions, and more particularly, but not exclusively, to methods for attenuating age-related symptoms or diseases comprising administrating such compositions to a subject. In some embodiments, subject is afflicted with chronic ischemia. In some embodiments, compositions and methods of the present invention are directed to attenuating age-related symptoms or diseases in a subject before chronic ischemia is apparent.

The present invention is based, in part, on the finding that controlled elevation of VEGF plasma levels attenuated symptoms associated with age-related symptoms and diseases in mice. Specifically, mildly increased VEGF signaling reduced muscle wasting and muscle mass loss, reduced pancreatic and hepatic steatosis, reduced subdermal fat loss, reduced weight gain during adulthood, induced and maintained brown adipose tissue, reduced age-related bone loss and fragility and attenuated age-related whole-body weight loss. Simultaneously, improved motor and coordination activity and prolonged life span were observed. The invention is further based, in part, on the finding that artificial mild increase of VEGF signaling by up to 3-fold, a therapeutic anti-aging response relevant for age-related symptoms and diseases can be achieved.

In some embodiments, the present invention is directed to methods and compositions for treating age-related symptoms or diseases, including but not limited to, in a subject afflicted with chronic ischemia. In some embodiments, the present invention is directed to methods and compositions for use in treating age-related symptoms or diseases, including but not limited to, in a subject e.g., before chronic ischemia is apparent. In some embodiments, the methods comprise increasing VEGF signaling by 3-fold at most in the subject, as compared to the VEGF baseline levels in the subject.

In some embodiments, the present invention is directed to methods for treating age-related symptoms or disease in a cell, tissue, organ or organism, the methods comprising administrating to the cell, tissue, organ or organism pharmaceutical composition of therapeutically effective amounts of VEGF-stimulating compounds.

In some embodiments, the present invention is directed to methods for treating age-related symptoms or disease or disorder in a subject in need thereof, the methods comprising administrating to the subject pharmaceutical composition of therapeutically effective amounts of VEGF receptor (VEGFR)-stimulating compounds, thereby increasing VEGF signaling by not more than 3-fold in the subject. In some embodiments, the present invention is directed to methods for treating age-related symptoms or disease or disorder in a subject in need thereof, the methods comprising administrating to the subject a pharmaceutical composition comprising therapeutically effective amounts of VEGF-stimulating compounds, VEGFR-stimulating compounds, or any combination thereof, thereby increasing VEGF signaling by not more than 3-fold in the subject. In some embodiments, the present invention is directed to methods for treating non-age-related wasting symptoms or disorders.

According to some embodiments, the invention is directed to compositions for increasing VEGF signaling by not more than 3-fold. In some embodiments, the composition comprises a VEGF-stimulating compound. In some embodiments, the composition comprises a VEGFR-stimulating compound. In some embodiments, the composition comprises a VEGF-stimulating compound, a VEGFR-stimulating compound or any combination thereof.

In some embodiments, the present invention is directed to methods and compositions for treating age-related symptoms. In some embodiments, age-related symptoms can be collectively termed ‘lesser ailments of aging’ (LAA). In some embodiments, LAA include, but are not limited to general muscle weakness, low temperature intolerance, age related cognitive decline including also minor memory lapses, skin wrinkles, and slow healing of bruises in the skin, wasting (total weight loss) muscle volume loss and bone density decrease.

In some embodiments, the present invention is directed to methods and compositions for treating age-related diseases or disorders. In some embodiments, age-related disease or disorder comprises any disease or disorder, incidence of which increases rapidly with age. In some embodiments, the term “rapidly” is exponentially. Non-limiting examples of age-related diseases include cardiovascular disease, cancer, arthritis, dementia, cataract, osteoporosis, metabolic diseases including diabetes, increased cholesterol and deterioration in lipid profile, hypertension, and neurodegenerative diseases including but not limited to Alzheimer's disease.

In one embodiment, “age-related” addresses a subject older than 50 years of age. In another embodiment, “age-related” addresses a subject older than 60 years of age. In another embodiment, “age-related” addresses a subject older than 70 years of age. In another embodiment, “age-related” addresses a subject older than 75 years of age. In another embodiment, “age-related” addresses a subject older than 80 years of age. In another embodiment, “age-related” addresses a subject older than 85 years of age. In another embodiment, “age-related” addresses a subject older than 90 years of age. In another embodiment, “age-related” addresses a to subject older than 95 years of age. In another embodiment, “age-related” addresses a subject older than 99 years of age.

In one embodiment, “age-related” addresses a subject older than 50-70 years of age, 60-80 years of age, 75-95 years of age, or 85-99 years of age. Each possibility represents a separate embodiment of the invention.

As used herein, the terms “age-related” and “age-associated” are interchangeable.

In some embodiments, the present invention is directed to methods and compositions used for treating whole body weight loss of the elderly. As defined herein, “weight loss” refers to the reduction of total body mass in a subject. In one embodiment, weight loss is a reduction of at least 3% of whole-body mass in a subject. In another embodiment, weight loss is a reduction of at least 4% of whole-body mass in a subject. In another embodiment, weight loss is a reduction of at least 5% of whole-body mass in a subject. In another embodiment, weight loss is a reduction of at least 10% of whole-body mass in a subject. In another embodiment, weight loss is a reduction of at least 20% of whole-body mass in a subject. In another embodiment, weight loss is a reduction of at least 30% of whole-body mass in a subject. In another embodiment, weight loss is a reduction of at least 40% of whole-body mass in a subject.

In some embodiments, the present invention is directed to methods and compositions used for treating muscle mass reduction of the elderly. As used interchangeably herein, the terms “muscle mass loss” and “wasting” refer to catabolism and/or the progressive loss of weight in a subject, or to loss of muscle mass and/or its progressive weakening and degeneration. In some embodiments, muscle wasting includes, but not limited to, sarcopenia and cachexia. In some embodiments, wasting may be due to a chronic or acute condition, such as ischemia (i.e. persisting over a long period of time), and may be associated with neurological, genetic or infectious pathologies, diseases, illnesses or conditions, including but not limited to, cardiac cachexia, cancer cachexia, malnutrition, diabetes, renal disease, cancer, end stage renal failure, andropause, frailty, emphysema, osteomalacia or cardiomyopathy. In some embodiments, wasting may include muscle wasting, for example as occurs with muscular dystrophies. If left unabated, wasting can have dire health consequences. For example, the changes that occur during wasting can lead to a weakened physical state that is detrimental to an individual's health, resulting in increased susceptibility to infection, or other diseases or conditions. In addition, muscle wasting is a strong predictor of morbidity and mortality in patients suffering from cachexia.

In another embodiment, muscle mass loss may be assessed by whole body dual energy X-ray absorptiometry scan examinations. As would be apparent to one of ordinary skill in the art, the obtained data includes values for bone mineral content (gr), bone mineral density (gr/cm²), fat mass (gr), lean mass (including bone mineral content [gr]), and fat percent for whole body and anatomical regions. From this data, appendicular skeletal mass (ASM) [kg]) can be calculated by summing the muscle masses of the four limbs, assuming that all non-fat and non-bone mass is skeletal muscle. In another embodiment, skeletal muscle index (SMI) is defined as the ratio of ASM/height (m²).

In some embodiments, the present invention is directed to methods and compositions used for treating age-related cognitive disease or disorder. As defined herein, “age-related cognitive impairment” refers to minor yet observable and measurable deterioration in cognitive abilities of the elderly, including but not limited to, study and thinking skills and memory. In one embodiment, elderly or aged patients with cognitive impairment have greater than normal difficulty performing complex daily tasks and learning, but without the inability to perform normal social everyday-and/or professional-functions typical of patients with Alzheimer's disease, or other similar neurodegenerative disorders eventually resulting in dementia. In one embodiment, cognitive impairment is characterized by subtle, clinically manifest deficits in cognition, memory, and functioning, amongst other impairments, which are not of sufficient magnitude to fulfill criteria for diagnosis of Alzheimer's disease or other dementia.

In some embodiments, the present invention is directed to methods and compositions for treating age-related kyphosis. As defined herein, the term “kyphosis” refers to a condition of the thoracic region of the spinal column where a dorsally exaggerated curvature is observed, possibly due to age-related reduction in muscle mass or due to osteoporosis. In one embodiment, kyphosis is characterized by a rounded upper back, or in extreme cases, a ‘hump-back’. In another embodiment, kyphosis is attributed to weakness of the spinal extensor musculature, wherein these muscles include the erector spinae (iliocostalis, longissimus and spinalis), thoracis, interspinales and the multifidus.

In another embodiment, as would be apparent to one of ordinary skill in the art, assessment of thoracic kyphosis is performed by standing lateral spine radiographs. In some embodiments, spinal radiographs may be taken in the supine position for comfort. The Cobb's angle of kyphosis is calculated from perpendicular lines drawn on a standard thoracic spine radiograph: a line extends through the superior endplate of the vertebral body, marking the beginning of the thoracic curve (usually at T4), and the inferior endplate of the vertebral body, marking the end of the thoracic curve (usually at T12). In another embodiment, as would be apparent to one of ordinary skill in the art, acceptable alternatives for assessment of thoracic kyphosis include, but are not limited to, the Debrunner kyphometer and the flexicurve ruler, both performed standing. In another embodiment, kyphosis index is calculated as the width divided by the length of the thoracic curve, multiplied by 100. In another embodiment, a kyphosis index value greater than 13 defines hyperkyphosis. In another embodiment, the terms “kyphosis” and “hyperkyphosis” are used herein interchangeably.

In some embodiments, the present invention is directed to methods and compositions for treating an age-related metabolic bone disease or disorder. As used herein, the term “bone perfusion” refers to blood and lymphatic flows through the circulatory and lymphatic systems, respectively, to the bone which characterizes bone remodeling (i.e., bone growth). In one embodiment, bone perfusion level serves as an indicator for bone plasticity. In another embodiment, bone perfusion level serves as an indicator of bone viability. In another embodiment, bone perfusion level serves as an indicator of bone remodeling capabilities. In another embodiment, bone perfusion level serves as an indicator of bone growth. In some embodiments, bone perfusion is reduced in the elderly. In some embodiments, reduced bone perfusion is indicative of a metabolic bone disease.

In one embodiment, bone perfusion may be assessed by positron emission tomography (PET) imaging. As would be apparent to one of ordinary skill in the art, Fluoride is incorporated into hydroxyapatite crystal of the bone to form fluorapatite (Temmerman et al., (2008)). This net uptake of Fluoride to the bone mineral compartment, termed the net fluoride influx rate (Kⁱ), is indicative of the extent of bone formation and mineralization, which can be quantified by the PET ligand ¹⁸F-Fluoride. As would be apparent to one of ordinary skill in the art, bone perfusion is assessed by immunostaining using bone formation markers. Non-limiting examples of bone formation markers include, but in not limited to cluster of differentiation 31 (CD31), endomucin (EMCN), total Alkaline Phosphatase (ALP), Bone Alkaline Phosphatase (B-ALP), Osteocalcin (OC, BGP), C-terminal propeptide of type I procollagen (PICP), N-terminal propeptide of type I procollagen (PINP), and others.

As used herein, the term “metabolic bone disease” encompasses, osteoporosis, Paget's disease, rachitis, osteomalacia, renal osteodystrophy of renal failure patients, hypoparathyroidism, and hyperparathyroidism.

In some embodiments, the present invention is directed to methods and compositions for treating age-related pancreatic disease or disorder. Non-limiting examples of age-related pancreatic disease include Diabetes mellitus type 2, pancreatic steatosis, acute pancreatitis and others. As used herein, “pancreatic steatosis” refers to the accumulation of fat in the pancreatic gland (i.e., the pancreas). Pancreatic steatosis may also be known as: pancreatic lipomatosis, fatty replacement, fatty infiltration, fatty pancreas, lipomatous pseudohypertrophy and non-alcoholic fatty pancreatic disease, among others.

In one embodiment, pancreatic steatosis may be assessed by an imaging method. Non-limiting examples of method applicable in assessing pancreatic steatosis include, but are not limited to, standard histology, ultrasonography, computed tomography (CT) and magnetic resonance imaging (MRI), among others.

As would be apparent to one of ordinary skill in the art, at least three methods are capable at quantifying pancreatic fat accumulation using MRI: (a) frequency shift between fat and water resonances; (b) the Dixon method; and (c) spectral-spatial excitation technique.

As used herein, the terms “acute pancreatitis”, refers to inflammation of the pancreas that occurs when digestive enzymes leak out of their collecting ducts and damage the surrounding tissue. In one embodiment, in acute pancreatitis digestive enzymes are released in their activated form from the exocrine portions of the pancreas, thereby causing inflammation, injury, autolysis and necrosis to the organ (i.e., the pancreas). In another embodiment, acute pancreatitis results in hemorrhage and pseudocyst formation within the gland. Common symptoms of acute pancreatitis include, but are not limited to, severe upper abdominal pain, nausea and vomiting.

In some embodiments, the present invention is directed to methods and compositions for treating age-related liver disease or disorder. As defined herein, the term “age-related fatty liver disease (FLD)” refers to a liver condition that occurs when lipids accumulate in hepatocytes (i.e. liver cells) and further impair hepatic microvascular circulation. In one embodiment, FLD can progress to more advanced liver disease such as nonalcoholic steatohepatitis (NASH; metabolic steatohepatitis). In one embodiment, NASH may progress to further liver damage ultimately leading to chronic liver failure and, in some cases, hepatocellular carcinoma.

Fatty liver diseases can be diagnosed in a subject by multiple methods. As would be apparent to one of ordinary skill the art, methods for diagnosing FLD include, but are not limited to, physical examination, blood test, imaging, tissue biopsy, or a combination thereof. In one embodiment, physical examination for detecting fatty liver in a subject includes seeking for an enlarged liver. In another embodiment, physical examination for detecting fatty liver in a subject includes examination of the subject's medical history of alcohol use, medication use, supplement use, or a combination thereof. In one embodiment, blood test for detecting fatty liver in a subject includes measuring the concentrations of liver enzymes, such as, but not restricted to, aspartate transaminase (AST), and alanine transaminase (ALT). In another embodiment, concentration of liver enzymes may be represented by the calculated ratio of AST to ALT, as would be apparent to one of ordinary skill in the art. In one embodiment, imaging methods for detecting fatty liver in a subject include any one of ultrasound, computational tomography (CT), or magnetic resonance imaging (MRI). In one embodiment, detecting fatty liver in a subject comprises collection of a tissue biopsy. In another embodiment, detecting fatty liver in a subject's tissue biopsy comprises sectioning, staining, or both. In another embodiment, one skilled in the art will appreciate that specific markers may be employed for the detection of specifically expressed genes and products thereof, instead of a general chemical stain (i.e., acidophilic stain, basophilic stain, charge-based stain, and the like).

In some embodiments, at least one age-related symptom or disease is selected for treatment from weight loss, weight gain, muscle mass loss, muscle atrophy, muscle wasting, cognitive impairment, kyphosis, hyperkyphosis, metabolic bone diseases, pancreatic steatosis, pancreatic inflammation, fatty liver disease, hepatosteatosis, hepatic inflammation, intestinal diseases, and brown adipose tissue (BAT) disease or condition.

As used herein, the term “brown adipose tissue” disease encompasses any condition in which the BAT manifests a reduced, inhibited, or impaired activity, functionality, structure, or any combination thereof.

The terms “brown adipose tissue” and “beige adipose tissue” are interchangeable.

As defined herein, the term “chronic ischemia” refers to a prolonged reduction in blood supply to cells and tissues of the body, resulting in poor but not full deprivation of oxidation. In some embodiments, a prolonged reduction in blood supply to cells and tissues of the body, is due to reduction of blood vessels number or deterioration of the blood vessels. In some embodiments, reduction of blood vessels number or deterioration of the blood vessels, results in reduction of supplemented angiocrine factors. In some embodiments, a prolonged reduction in blood supply to cells and tissues of the body, results in reduction of supplemented angiocrine factors. As defined herein, the term “angiocrine factor” refers to any molecule produced by an endothelial cell, which stimulates an organ-specific repair activity in a damaged organ or maintains homeostasis in a non-injured organ. The terms “angiocrine factor” and “angiocrine growth factor” are interchangeable. In some embodiments, a prolonged reduction in blood vessels density and quality in tissues of the body, results in differential composition of supplemented angiocrine factors. In some embodiments, angiocrine factors are essential for the maintenance of an organ homeostasis. In some embodiments, chronic ischemia refers to poor perfusion. Non-limiting examples of chronic ischemia may include ischemic heart disease, ischemic colitis, mesenteric colitis, vascular dementia, and others.

In some embodiments, chronic ischemia results in acidosis. In some embodiments, acidosis is metabolic acidosis. In some embodiments, metabolic acidosis is lactate acidosis. In some embodiments, chronic ischemia is detected based on acidosis. In some embodiments, in normal blood sample, lactate levels are lower than 1.8 mmol/L. In some embodiments, hyperlactatemia is having blood lactate levels greater than 1.8 mmol/L and lower than 5 to 6 mmol/L. In some embodiments, lactic acidosis is having blood pH lower than 7.35 and lactate levels greater than 5-6 mmol/L.

In some embodiments, the present invention is directed to methods and compositions capable of reducing lactic acidosis. In one embodiment, VEGF stimulating- and VEGFR stimulating-compounds of the invention reduce lactate blood levels to lower than 5-6 mmol/L, 4-5 mmol/L, 3-4 mmol/L, 2-3 mmol/L, or lower than 2 mmol/L. Each possibility represents a separate embodiment of the invention. In some embodiments, VEGF stimulating- and VEGFR stimulating-compounds of the invention reduce lactate blood levels by at least 5%, 10%, 20%, 35%, 50%, 65%, 80%, 90%, or 99%, or any value and range therebetween. Each possibility represents a separate embodiment of the invention. In some embodiments, VEGF stimulating- and VEGFR stimulating-compounds of the invention reduce lactate blood levels by 1-5%, 4-10%, 8-20%, 15-35%, 20-50%, 40-65%, 55-80%, 70-90%, or 85-99%. Each possibility represents a separate embodiment of the invention. In some embodiments, VEGF stimulating- and VEGFR stimulating-compounds of the invention reduce lactate blood levels by at least 2-fold, 3-fold, 5-fold, or 10-fold, or any value and range therebetween. Each possibility represents a separate embodiment of the invention.

In some embodiments, chronic ischemia results in liver damage. In some embodiments, liver damage is indicated by an increase in serum aminotransferases levels. In some embodiments, aminotransferases comprise aspartate transaminase (AST), and alanine transaminase (ALT). In some embodiments, normal AST blood levels are 10-40 IU/L. In some embodiments, ALT normal blood levels are 7-56 IU/L. A mild increase of aminotransferases blood levels is defined herein as elevation of 2-3-fold. In some embodiments, ischemia-indicative serum levels of an aminotransferase can reach levels of 1,000 IU/L to 3,000 IU/L, or any value and range therebetween.

In one embodiment, a VEGF stimulating- or a VEGFR stimulating-compound reduces AST or ALT blood levels to lower than 3,000 IU/L, 2,000 IU/L, 1,000 IU/L, 500 IU/L, 100 IU/L, or lower than 60 IU/L, or any value and range therebetween. Each possibility represents a separate embodiment of the invention. In some embodiments, a VEGF stimulating- or a VEGFR stimulating-compound reduces AST or ALT blood levels by at least 5%, 10%, 20%, 35%, 50%, 65%, 80%, 90%, or 99%, or any value and range therebetween. Each possibility represents a separate embodiment of the invention. In some embodiments, a VEGF stimulating- or a VEGFR stimulating-compound reduces AST or ALT blood levels by 1-5%, 3-10%, 6-20%, 16-35%, 20-50%, 30-65%, 45-80%, 60-90%, or 80-99%. Each possibility represents a separate embodiment of the invention. In some embodiments, a VEGF stimulating- or a VEGFR stimulating-compound reduces AST or ALT blood levels by at least 2-fold, 10-fold, 50-fold, 100-fold, 250-fold, 400-fold, or 500-fold, or any value and range therebetween. Each possibility represents a separate embodiment of the invention.

Methods for determining the levels of lactate, AST, or ALT are common, and would be apparent to one of ordinary skill in the art. In some embodiments, the levels of lactate, AST, or ALT are quantified in a subject's sample. In some embodiments, the sample comprises a bodily fluid. In some embodiments, the sample comprises a tissue or a fragment thereof. In some embodiments, the bodily fluid comprises blood, serum, or plasma.

As used herein, the terms “treatment” or “treating” of a disease, disorder, or condition encompasses alleviation of at least one symptom thereof, a reduction in the severity thereof, or inhibition or slowing of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient. In some embodiments, treatment refers to a clinical application of active agents to combat an already existing condition whose clinical presentation has yet to be realized in a patient.

As used herein, the term “condition” includes anatomic and physiological deviations from the normal that constitute an impairment of the normal state of the living animal or one of its parts, that interrupts or modifies the performance of the bodily functions.

Any concentration ranges, percentage range, or ratio range recited herein are to be understood to include concentrations, percentages or ratios of any integer within that range and fractions thereof, such as one tenth and one hundredth of an integer, unless otherwise indicated.

In one embodiment, methods of this invention are for treating age-related symptoms and diseases or disorders. In one embodiment, the methods comprise compositions comprising a safe and effective amount of the stimulating compounds, which are used for attenuating age-related diseases and disorders. In another embodiment, methods on the present invention are used in an anti-aging treatment in a subject in need thereof. In another embodiment, methods of the present invention are used in a rejuvenating treatment in a subject in need thereof.

In one embodiment, the methods of the invention are for inducing longevity in a subject. In another embodiment, the methods of the invention are for inducing life extension in a subject. In one embodiment, the methods of the invention extend life span of a subject by at least 5%, by at least 10%, by at least 20%, by at least 30%, by at least 35%, by at least 40%, or by at least 50%, or any value and range therebetween. Each possibility represents a separate embodiment of the invention.

In another embodiment, methods of the invention are directed to extend life span of a subject by at least one year, by at least 5 years, by at least 10 years, by at least 15 years, or by at least 25 years, or any value and range therebetween. Each possibility represents a separate embodiment of the invention.

In some embodiments, methods of the invention are directed to maintain VEGF plasma levels over a time of period. As used herein, the term “maintain” refers to keeping at a relatively constant level. In some embodiments, “maintain” is keeping a constant level on average across time (e.g. at least one day, at least one week, and at least one month). In one embodiment, a constant level comprises equilibrium. In one embodiment, a constant level comprises a steady state. In some embodiments, maintained levels are fluctuating across time. As used herein, the term “fluctuating” comprises an increase and subsequent decrease, or decrease and subsequent increase, by not more than 0.1%, 0.5%, 1%, 5%, 10%, 20%, 30%, 40%, 50%, 60%, 85% or 90% across time, or any value and range therebetween. Each possibility represents a separate embodiment of the invention. As used herein, fluctuating comprises an increase and subsequent decrease, or decrease and subsequent increase by 0.1-0.5%, 0.4-2%, 1-5%, 4-10%, 9-20%, 15-30%, 25-40%, 30-50%, 40-60%, 55-85%, or 80-100% across time. Each possibility represents a separate embodiment of the invention.

In some embodiments, methods of the invention are directed to maintain 3-fold increased VEGF signaling compared to a baseline in a subject for at least 1 day, at least 7 days, at least 30 days, at least 1 month, at least 6 months, at least 1 year, or any value and range therebetween. Each possibility represents a separate embodiment of the invention. In one embodiment, methods of the invention are directed to maintain 3-fold increased VEGF signaling indefinitely. In another embodiment, indefinitely comprises an everlasting effect. In one embodiment, indefinitely is everlasting.

In one embodiment, the methods comprise administering a stimulating compound of to a subject in need thereof, at least once a week, at least once a month, at least once every other month, at least once in 6 months, at least twice in 6 months, or at least once a year, or any value and range therebetween. Each possibility represents a separate embodiment of the invention. In another embodiment, the methods comprise administering stimulating compounds of the invention to a subject in need thereof only once.

In some embodiments of the methods described herein, inhibiting is reducing by more than 2%, by more than 5%, by more than 10%, by more than 25%, by more than 50%, by more than 75%, by more than 90%, by more than 95%, or by more than 99%, or any value and range therebetween. Each possibility represents a separate embodiment of the invention.

As used herein, the terms “attenuate”, “inhibit”, “revert”, and “reverse” are interchangeable.

As used herein, the terms “anti-aging”, and “rejuvenating” are interchangeable.

As used herein, the terms “life span”, “life expectancy”, and “life duration” are interchangeable.

### VEGF, VEGFR and Stimulating Compounds

As used herein, VEGF refers to the “vascular endothelial growth factor”. In one embodiment, VEGF is VEGF-A (accession number NP 001303939.1). In one embodiment, VEGF is VEGF-B (accession no. NP 003368.1). In one embodiment, VEGF is VEGF-C (accession no. AAH63685.1). In one embodiment, VEGF is VEGF-D (accession on. CAA03942.1). In one embodiment, VEGF is VEGF-E (accession nos. GNN_A; 2GNN_B; 2GNN_C; or 2GNN_D). In one embodiment, VEGF is VEGF-F (accession nos. ACN22043.1 or JAC96562.1). In one embodiment, VEGF-E and VEGF-F are VEGF-related proteins. In one embodiment, VEGF is a human VEGF. In another embodiment, VEGF is human VEGF-A. In another embodiment, human VEGF-A is comprised of 5 isoforms resulting of alternative splicing of mRNA encoding 121, 145, 165, 189 or 206 amino acids in length (VEGF₁₂₁₋₂₀₆), all of which are capable of stimulating mitogenesis in endothelial cells.

As used herein, VEGFR refers to the “vascular endothelial growth factor receptor”. In some embodiments, VEGFR is selected from the group consisting of: VEGFR-1, VEGFR-2, VEGFR-3 or VEFG accessory receptor. In one embodiment, VEGFR-1 is FM (accession no. P17948.2). In another embodiment, VEGFR-2 is Kdr/Flk-1 (accession no. P35968.2). In another embodiment, VEGFR-3 is Flt4 (accession no. P35916.3). In one embodiment, VEGFR-like receptor is Neuropilin-1 (NRP-1; accession no. NM_003873.5). In another embodiment, VEGFR-like receptor is Neuropilin-2 (NRP-2; accession no. NM_201266.1). In another embodiment, the terms “VEGFR” and “VEGFR-like-receptor” are interchangeable.

As used herein, VEGF signaling refers to biological actions of VEGF, which are mediated through specific binding with its designated cell-associated family of receptors. In one embodiment VEGF signaling refers to biological actions of VEGF, which are mediated through VEGFR-1. In one embodiment, VEGF signaling refers to biological actions of VEGF, which are mediated through VEGFR-2. In one embodiment, VEGF signaling refers to biological actions of VEGF, which are mediated through VEGFR-3. In one embodiment, VEGF-A signaling is propagated predominantly through interactions with VEGFR-1 and VEGFR-2. In one embodiment, VEGF-B signaling is propagated predominantly through interactions with VEGFR-1. In one embodiment, VEGF-C signaling is propagated predominantly through interactions with VEGFR-2 and VEGFR-3. In one embodiment, VEGF-D signaling is propagated predominantly through interactions with VEGFR-3. In one embodiment, VEGF-E signaling is propagated predominantly through interactions with VEGFR-2. In another embodiment, VEGFR binds VEGF with dissociation constant between 10⁻¹¹ M to 10⁻¹² M. In some embodiments, increased VEGF signaling comprises increased number of VEGF-VEGFR complexes. In some embodiments, increased VEGF signaling comprises increased number or amount of free- or circulating-VEGF.

As defined herein, VEGF signaling governs vasculogenesis. In one embodiment, VEGF signaling governs angiogenesis or any other vascular cell-related function. In one embodiment, VEGF signaling governs osteogenesis. In another embodiment, VEGF signaling affects non-vascular cells.

As defined herein, “VEGF-stimulating compound” refers to any molecule that specifically enhances VEGF signaling. In some embodiments, enhancing VEGF signaling includes contacting a polynucleotide comprising a VEGF encoding sequence and inducing its expression, thereby resulting in its elevated levels. In some embodiments, elevated levels are increased levels of the VEGF encoding gene transcription. In one embodiment, a VEGF-stimulating compounds that increase its transcription includes Hif1, a Hif1 stabilizer, or both. In some embodiments, elevated levels are increased levels of the VEGF mRNA molecules. In some embodiments, elevated levels of VEGF transcription are induced by hypoxia. In some embodiments, elevated levels of VEGF transcription are induced by hypoxia-mimetics. In some embodiments, elevated levels are increased levels of the VEGF mRNA translation. In some embodiments, elevated levels are increased VEGF mRNA stability. In one embodiment, a VEGF-stimulating compound inhibits VEGF-specific regulatory RNA. In one embodiment, a VEGF-stimulating compound inhibits VEGF-specific microRNA. In one embodiment, a VEGF-stimulating compound is an antagomiR. In some embodiments, elevated levels are increased levels of the VEGF polypeptide. In one embodiment, a VEGF-stimulating compound increases VEGF secretion. In one embodiment, a VEGF-stimulating compound increases VEGF protein stability. In one embodiment, a VEGF-stimulating compound increases VEGF biological half-life (t_{1/2}) in the circulatory system. In one embodiment, a VEGF-stimulating compound increases the number of free VEGF polypeptide molecules in the circulatory system. As used herein, the term “free” refers to a polypeptide that is unbound by a chaperone, a binding protein, a carrying protein, a receptor, a soluble receptor, an antibody, or any peptide or polypeptide having specific binding affinity to the VEGF polypeptide. In one embodiment, a VEGF-stimulating compound inhibits VEGF-specific proteolysis. In one embodiment, a VEGF-stimulating compound inhibits VEGF-specific proteases. In one embodiment, a VEGF-stimulating compound is a VEGF-specific protease inhibitor. In one embodiment, a VEGF-stimulating compound is a VEGF analogue. In one embodiment, VEGF-stimulating compound is a VEGF partial polypeptide or a derivative peptide thereof. In one embodiment, a VEGF-stimulating compound is a VEGF peptidomimetic compound. In some embodiments, a VEGF peptidomimetic compound is characterized by specific binding affinity to an inhibitory soluble VEGF decoy receptor. In some embodiments, a VEGF peptidomimetic compound has greater binding affinity to an inhibitory soluble VEGF decoy receptor (sVEGFR), compared to the binding affinity of the VEGF polypeptide to the inhibitory soluble VEGF decoy receptor. In one embodiment, an inhibitory soluble VEGF decoy receptor is sVEGFR1 or sVEGFR2. In one embodiment, the VEGF-stimulating compound is a sVEGFR inhibitor. In some embodiments, the VEGF-stimulating compound binds or inhibits sVEGFR. In some embodiments, a specific sVEGFR is any reagent that modifies alternative splicing modes of the VEGFR, RNA-based molecule that binds selectively to soluble VEGFR mRNA, or a protease cleaving an extracellular part or portion of a VEGFR. In one embodiment, a VEGF-stimulating compound is an extracellular matrix degrading enzyme. In one embodiment, an extracellular matrix degrading enzyme is low molecular weight heparins. In one embodiment, a VEGF-stimulating compound increases binding affinity of VEGF to its family of receptors. In another embodiment, a VEGF-stimulating compound is any small molecule capable of stimulating VEGF signaling. In some embodiments, a small molecule comprises a dense negatively charged molecule. In some embodiments, elevated levels of the VEGF polypeptide are achieved by a transfection of a vector or a plasmid. In some embodiments, the vector comprises a polynucleotide comprising a VEGF encoding polynucleotide sequence. In some embodiments, the increased levels of the VEGF encoding gene are induced by VEGF gene editing. In some embodiments, gene editing is a molecular alteration in the VEGF genomic polynucleotide sequence inducing the gene's over-expression. In some embodiments, the gene editing is achieved by Clustered Regularly Interspaced Short Palindromic Repeats (CRISPR) system. In some embodiments, a VEGF-stimulating compound is a molecule capable of specifically inhibiting a VEGF-inhibitor. In some embodiments, a VEGF-stimulating compound is a VEGF B inhibitor. In some embodiments, a VEGF-stimulating compound is a VEGF C inhibitor. In some embodiments, a VEGF-stimulating compound is placental growth factor (PIGF; accession number P49763).

As used herein, a “VEGFR-stimulating compound” refers to any molecule that specifically enhances VEGFR signaling. In one embodiment, a VEGFR-stimulating compound increases VEGFR transcription. In one embodiment, a VEGFR-stimulating compound increases VEGFR mRNA stability. In one embodiment, a VEGFR-stimulating compound inhibits a VEGFR-specific regulatory RNA. In one embodiment, a VEGFR-stimulating compound inhibits a VEGFR-specific microRNA. In one embodiment, a VEGFR-stimulating compound is an antagomiR. In one embodiment, a VEGFR-stimulating compound enhances VEGFR translation. In one embodiment, a VEGFR-stimulating compound increases VEGFR transport to the cell membrane. In one embodiment, a VEGFR-stimulating compound increases VEGFR protein stability. In one embodiment, a VEGFR-stimulating compound increases VEGFR biological half-life (t_{1/2}). In one embodiment, a VEGFR-stimulating compound increases VEGFR protein turn-over. In one embodiment, a VEGFR-stimulating compound inhibits VEGFR-specific proteolysis. In one embodiment, a VEGFR-stimulating compound inhibits a VEGFR-specific protease. In one embodiment, a VEGFR-stimulating compound is a VEGFR-specific protease inhibitor. In one embodiment, a VEGFR-stimulating compound is a VEGF analogue. In one embodiment, a VEGFR-stimulating compound is an endogenous agonist. In one embodiment, a VEGFR-stimulating compound is an exogenous agonist. In one embodiment, a VEGFR-stimulating compound is a synthetic agonist. In one embodiment, a VEGFR-stimulating compound is a partial agonist. In one embodiment, a VEGFR-stimulating compound is a full agonist. In one embodiment, a VEGFR-stimulating compound is a super agonist. In one embodiment, a VEGFR-stimulating compound is a VEGF partial or derivative peptide thereof. In one embodiment, a VEGFR-stimulating compound is a VEGF peptidomimetic compound. In one embodiment, a VEGFR-stimulating compound increases binding affinity of VEGFR to its family of ligands. In another embodiment, a VEGFR-stimulating compound is any small molecule capable of stimulating VEGFR signaling. In some embodiments, a VEGFR-stimulating compound is a molecule capable of inhibiting a VEGFR inhibitor.

In one embodiment, a controlled increase of VEGF levels is increasing standard VEGF levels by at least 5%, by at least 10%, by at least 25%, by at least 50%, by at least 75%, by at least 100%, by at least 150%, by at least 175%, by at least 200%, by at least 225%, by at least 300%, or by at least 1,000%, or any value and range therebetween. Each possibility represents a separate embodiment of the invention.

In one embodiment, a controlled increase of VEGF levels is increasing standard VEGF levels by 5-10%, 4-10%, 10-25%, 20-50%, 35-75%, 60-100%, 75-150%, 120-175%, 150-200%, 170-225%, 200-300%, 275-1,000%. Each possibility represents a separate embodiment of the invention.

In one embodiment, a controlled increase of VEGF levels is increasing standard VEGF levels by at least 1.05-fold, by at least 1.1-fold, by at least 1.25-fold, by at least 1.5-fold, by at least 1.75-fold, by at least 2-fold, by at least 2.25-fold, by at least 3-fold, by at least 3.25-fold, by at least 3.5-fold, by at least 5-fold, or by at least 10-fold, or any value and range therebetween. Each possibility represents a separate embodiment of the invention.

In some embodiments, methods of the present invention further comprise a step of detecting VEGF state in a subject by determining the plasma levels of VEGF in the subject.

In one embodiment, standard VEGF serum level ranges from 1-100 pg/ml. In one embodiment, standard VEGF serum level ranges from 2-250 pg/ml. In one embodiment, standard VEGF serum level ranges from 5-500 pg/ml. In one embodiment, standard VEGF serum level ranges from 5-750 pg/ml. In one embodiment, standard VEGF serum level ranges from 10-1,000 pg/ml. In one embodiment, standard VEGF serum level ranges from 150-1,500 pg/ml. In one embodiment, standard VEGF serum level ranges from 500-2,500 pg/ml. In another embodiment, the term “standard” used herein, is interchangeable with any of “normal”, “regular” “proper”, “naïve” or “healthy”.

In some embodiments, standard VEGF plasma level ranges from 1 to 250 pg/ml. In one embodiment, standard VEGF plasma level ranges from 1-20 pg/ml. In one embodiment, standard VEGF plasma level ranges from 15-25 pg/ml. In one embodiment, standard VEGF plasma level ranges from 20-40 pg/ml. In one embodiment, standard VEGF plasma level ranges from 25-50 pg/ml. In one embodiment, standard VEGF plasma level ranges from 35-75 pg/ml. In one embodiment, standard VEGF plasma level ranges from 50-100 pg/ml. In one embodiment, standard VEGF plasma level ranges from 75-250 pg/ml.

The term “determining” is used in the broadest sense, including qualitative and quantitative determination of the target molecule. In one embodiment, the determining step described herein is only used to identify the presence of VEGF in a biological sample. In another embodiment, the determining step is used to detect levels of VEGF in specimens. In yet another embodiment, the determining step can be used to quantify the amount of VEGF in at least one sample, and further compare VEGF levels between different samples.

In some embodiments, VEGF levels can be determined in a biological sample by any method known to one of ordinary skill in the art, Non-limiting examples for such determination methods include, but are not limited to, immunoassays (e.g., enzyme-linked immunosorbent assay (ELISA), sandwich ELISA, immunohistochemistry, immunocytochemistry, etc.), polymerase chain reaction (PCR) (e.g., quantitative PCR, RT-PCR, etc.), and others.

As used herein, the term “biological sample” refers to any type of physical specimen which has been obtained, collected, derived, dissected or any equivalent thereof, from an animal. In some embodiments, the biological sample comprises biological fluids selected from: serum, plasma, vitreous fluid, lymph fluid, synovial fluid, follicular fluid, seminal fluid, amniotic fluid, milk, whole blood, urine, cerebrospinal fluid, saliva, sputum, tears, perspiration, mucus, or tissue culture media, or a combination thereof. Each possibility represents a separate embodiment of the invention. In some embodiments, the biological sample is selected from: tissue extracts, homogenized tissue, cellular extracts, or a biopsy, or a combination thereof. Each possibility represents a separate embodiment of the invention.

In another embodiment, biological sample is obtained from a mammal. In another embodiment, biological sample is obtained from a human.

Methods for obtaining a biological sample from an animal o a subject are common, and would be apparent to one of ordinary skill in the art.

In some embodiments of the methods described herein, the inhibitory nucleic acid is an antisense oligonucleotide.

As used herein, an “antisense oligonucleotide” refers to a nucleic acid sequence that is reversed and complementary to a DNA or RNA sequence, such as that of a microRNA.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g. the ‘seed sequence’), e.g., nts 2-7 or 20.

As well apparent to one skilled in the art, a CRISPR system used herein refers to the method allowing a CRISPR complex to bind to the polynucleotide such that the binding results in increased or decreased expression of the polynucleotide. In some embodiments, the method further comprises delivering one or more vectors to the cells, wherein the one or more vectors drive expression of one or more of: the CRISPR enzyme, the guide sequence linked to the tracer mate sequence, and the tracer sequence.

The inhibitory nucleic acids useful in the methods described herein have at least 80% sequence complementarity to a target region within the target nucleic acid, e.g., 90%, 95%, or 100% sequence complementarity to the target region within the targeted miR.

In some embodiments of the methods described herein, the inhibitory nucleic acid has one or more chemical modifications to the backbone or side chains as described herein. In some embodiments of the methods described herein, the inhibitory nucleic acid is an antagomir. In some embodiments of the methods described herein, the inhibitory nucleic acid has at least one locked nucleotide, and/or has a phosphorothioate backbone.

In some embodiments of the methods described herein, the inhibitory nucleic acid is an interfering RNA. In some embodiments, the interfering RNA is a small hairpin RNA (shRNA) or small interfering RNA (siRNA).

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

As used herein “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long.

As used herein, an “antagomir” refers to a small synthetic RNA having complementarity to a specific microRNA target, with either mispairing at the cleavage site or one or more base modifications to inhibit cleavage. In another embodiment, an “antagomir” refers to a small synthetic RNA having complementarity to a population of microRNA targets, with either mispairing at the cleavage site or one or more base modifications to inhibit cleavage.

As used herein, the phrase “post-transcriptional processing” refers to RNA processing that occurs after transcription and is mediated, for example by the enzymes Dicer and/or Drosha. in the case of miRNAs.

As used herein, a “peptide” refers to either a naturally or artificially manufactured short chain of amino acid monomers, which are linked to one another by means of amide (peptide) bonds. With this respect, a “polypeptide” is a long, continuous peptide polymer. Peptides and polypeptides may comprise 50 amino acids, 40 amino acids, 30 amino acids, 20 amino acids, or less than 10 amino acids. The terms “peptide”, “polypeptide” and “protein” used herein, are interchangeable.

“Peptide mimetics” or “peptidomimetics” are structures which serve as substitutes for peptides in interactions between molecules (Morgan et al., 1989). Peptide mimetics include synthetic structures which may or may not contain amino acids and/or peptide bonds but retain the structural and functional features of a peptide, or agonist or antagonist (i.e. enhancer or inhibitor) of the invention. Peptide mimetics also include peptoids, oligopeptoids (Simon et al., 1972); and peptide libraries containing peptides of a designed length representing all possible sequences of amino acids corresponding to a motif, peptide, or agonist or antagonist (i.e. enhancer or inhibitor) of the invention.

In one embodiment, the present invention provides a vector or a plasmid comprising the nucleic acid molecule as described herein. In one embodiment, a vector or a plasmid is a composite vector or plasmid. In one embodiment, a vector or a plasmid is a man-made vector or plasmid comprising at least one DNA sequence which is artificial. In one embodiment, the present invention provides a vector or a plasmid comparing: Adeno Associated Virus, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In one embodiment, the present invention provides a vector or a plasmid comprising regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMT010/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells. According to some embodiments, a recombinant adeno-associated vector (AAV) comprising one or more polynucleotide sequence encoding the VEGF, VEGF-stimulating compound, VEGFR-stimulating compound, or any combination thereof, is provided.

In some embodiments, the AAV encodes a VEGF. In some embodiments, the AAV encodes a VEGF-mimetic. In some embodiments, the AAV encodes a VEGF-stimulating compound. In some embodiments, the AAV encodes a VEGFR-stimulating compound.

In one embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A “fragment” of the peptide will preferably comprise less than the total amino acid sequence of the full native peptide; preferably the fragment retains its biological activity.

A “variant” of the peptide also refers to a peptide wherein at one or more positions there have been amino acid insertions, deletions, or substitutions, either conservative or non-conservative, provided that such changes result in a protein whose basic properties; protein interaction; thermostability; activity in a certain pH-range (pH-stability), have not significantly been changed. “Significantly” in this context means that one skilled in the art would say that the properties of the variant may still be different but would not be unobvious over the ones of the original protein.

The term “peptidomimetic” refers to a compound that mimics the conformation and desirable features of a particular peptide as a therapeutic agent, but that avoids the undesirable features. For example, morphine is a compound which can be orally administered, and which is a peptidomimetic of the peptide endorphin. There are a number of different approaches to the design and synthesis of peptidomimetics, as is well known in the art.

The term “agonist” may refer to a small molecule. The term “small molecule” is well known in pharmacology and biochemistry as a low molecular weight chemical compound. Many pharmaceutical drugs are small molecules. Such agonists may be identified as part of a high throughput screen of small molecule libraries.

### Pharmaceutical Compositions

According to some embodiments, there is provided a pharmaceutical composition comprising VEGF in an amount effective to increase VEGF signaling by 3-fold at most in a subject's plasma compared to a baseline.

In some embodiments, VEGF in an amount effective to increase VEGF signaling by 3-fold at most, reduces muscle weakness. In some embodiments, VEGF in an amount effective to increase VEGF signaling by 3-fold at most, reduces or improves cold intolerance. In some embodiments, VEGF in an amount effective to increase VEGF signaling by 3-fold at most, reduces skin wrinkles. In some embodiments, VEGF in an amount effective to increase VEGF signaling by 3-fold at most, reduce rate or period of skin healing. In some embodiments, VEGF in an amount effective to increase VEGF signaling by 3-fold at most, reduces weight loss in the elderly. In some embodiments, VEGF in an amount effective to increase VEGF signaling by 3-fold at most, reduces weight gain during adolescence. In some embodiments, VEGF in an amount effective to increase VEGF signaling by 3-fold at most, reduces cognitive impairment. In some embodiments, VEGF in an amount effective to increase VEGF signaling by 3-fold at most, reduces level of kyphosis, or kyphosis index. In some embodiments, VEGF in an amount effective to increase VEGF signaling by 3-fold at most, improves bone mineralization levels. In some embodiments, VEGF in an amount effective to increase VEGF signaling by 3-fold at most, reduces bone demineralization rate. In some embodiments, VEGF in an amount effective to increase VEGF signaling by 3-fold at most, reduces inhibition of brown adipose tissue activity. In some embodiments, VEGF in an amount effective to increase VEGF signaling by 3-fold at most, induces activation of brown adipose tissue activity. In some embodiments, VEGF in an amount effective to increase VEGF signaling by 3-fold at most, reduces subdermal fat loss.

In some embodiments, VEGF in an amount effective to increase VEGF signaling by 3-fold at most, is used for treating or preventing a muscle wasting disease. In some embodiments, VEGF in an amount effective to increase VEGF signaling by 3-fold at most, is used for treating or preventing osteoporosis. In some embodiments, VEGF in an amount effective to increase VEGF signaling by 3-fold at most, is used for treating or preventing a pancreatic disease. In some embodiments, VEGF in an amount effective to increase VEGF signaling by 3-fold at most, is used for treating or preventing intestinal disease. In some embodiments, VEGF in an amount effective to increase VEGF signaling by 3-fold at most, is used for treating or preventing occurrence of a neoplastic lesion or cancer. In some embodiments, VEGF in an amount effective to increase VEGF signaling by 3-fold at most, is used for treating or preventing a hepatic disease.

In some embodiments, VEGF in an amount effective to increase VEGF signaling by 3-fold at most, does not induce formation of a leaky blood vessel. In some embodiments, VEGF in an amount effective to increase VEGF signaling by 3-fold at most, inhibits, reduces or prevent the formation of a leaky blood vessel. As used herein, the term “blood vessel” encompasses a capillary, an arteriole, an arteria, an artery, a venule, a vena, a vein, or a sinusoid. In some embodiments, VEGF in an amount effective to increase VEGF signaling by 3-fold at most, does not induce cancer. In some embodiments, VEGF in an amount effective to increase VEGF signaling by 3-fold at most, does not induce oxidative stress. In some embodiments, VEGF in an amount effective to increase VEGF signaling by 3-fold at most, does not induce age-related disease of the eye. In some embodiments, VEGF in an amount effective to increase VEGF signaling by 3-fold at most, does not induce cataract formation. In some embodiments, VEGF in an amount effective to increase VEGF signaling by 3-fold at most, does not induce neovascular and non-exudative AMD-like pathologies. In some embodiments, VEGF in an amount effective to increase VEGF signaling by 3-fold at most, does not induce an age-related opacification in the lens, associated with: ERK hyperactivation, oxidative damage, increased expression of the NLRP3 inflammasome effector cytokine IL-1b, or a combination thereof. In some embodiments, VEGF in an amount effective to increase VEGF signaling by 3-fold at most, does not induce oxidative stress, or IL-1b expression, or both in the retinal pigment epithelium (RPE). In some embodiments, VEGF in an amount effective to increase VEGF signaling by 3-fold at most, does not induce excessive permeability, excessive neovascularization, pathological extramedullary hematopoiesis, or any combination thereof.

In one embodiment, compositions of the present invention comprise an anti-aging compound used for attenuating age-related diseases, disorders, or symptoms thereof in a subject. In one embodiment, the compositions comprise an anti-aging compound used for reverting age-related diseases and disorders. In another embodiment, the compositions comprise an anti-aging compound used for reversing age-related diseases and disorders. In one embodiment, the compositions comprise an anti-aging compound used for increasing life span. In one embodiment, the compositions comprise an anti-aging compound used for increasing life expectancy. In another embodiment, the compositions comprise an anti-aging compound used for extending life duration.

In some embodiments, the compositions comprise solutions or emulsions, which in some embodiments are aqueous solutions or emulsions comprising a safe and effective amount of the compounds of the present invention and optionally, other compounds, intended for topical intranasal administration. In some embodiments, the compositions comprise from about 0.01% to about 10.0% w/v of a subject compound, more preferably from about 0.1% to about 2.0, which is used for systemic delivery of the compounds by the intranasal route.

In some embodiments, the compositions of the invention further comprise an acceptable carrier or diluent. In some embodiments, the carrier or diluent is a pharmaceutically acceptable carrier or diluent. In some embodiments, the compositions of the invention are pharmaceutical compositions.

In another embodiment, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

In another embodiment, the pharmaceutical compositions are applied topically to body surfaces, and are thus formulated in a form suitable for topical administration or application. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, the pharmaceutical compositions are manufactured by processes well known in the art, e.g., by means of conventional mixing, dissolving, granulating, dragee-making, levigating, emulsifying, encapsulating, entrapping or lyophilizing processes.

In one embodiment, a pharmaceutical composition of the invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectable compositions are formulated in aqueous solutions. In one embodiment, injectable compositions are formulated in physiologically compatible buffers such, but not limited to Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multi-dose containers with optionally, an added preservative. In some embodiments, the compositions comprise suspensions, solutions or emulsions in oily or aqueous vehicles, and comprise formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcysteine, sodium metabisulfite and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration comprise aqueous solutions of the active preparation in water-soluble form. Additionally, suspensions of the active ingredients, in some embodiments, are prepared as appropriate oily or water-based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances which increase the viscosity of the suspension, such as sodium carboxymethyl cellulose, sorbitol or dextran. In another embodiment, the suspension further comprises suitable stabilizers or agents which increase the solubility of the active ingredients to allow for the preparation of highly concentrated solutions.

In another embodiment, the active compound can be delivered in a vesicle or particularly in a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., (1980); Saudek et al., (1989). In another embodiment, polymeric materials can be used. In yet another embodiment, a controlled release system can be placed in proximity to the therapeutic target, thus requiring only a fraction of the systemic dose (see, e.g., Goodson, in Medical Applications of Controlled Release, supra, vol. 2, pp. 115-138 (1984). Other controlled release systems are discussed in the review by Langer (1990).

In some embodiments, the active ingredient is in a powder form for constitution with a suitable vehicle, e.g., sterile, pyrogen-free water-based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975)].

In one embodiment, dosing can be of a single or a plurality of administrations, with course of treatment lasting from several days to several weeks or until cure is affected or diminution of the disease state is achieved. In another embodiment, dosing can depend on severity and responsiveness of the condition to be treated.

In one embodiment, the amount of a composition to be administered will, of course, be dependent on the subject being treated, the severity of the affliction, the manner of administration, the judgment of the prescribing physician, etc.

In one embodiment, a composition including the preparation of the present invention formulated with a compatible pharmaceutical carrier is also prepared, placed in an appropriate container, and labeled for treatment of an indicated condition.

In some embodiment, the term “therapeutically effective amount” refers to a concentration of a VEGF-, VEGFR-stimulating compound, or any combination thereof, effective to treat a disease or disorder in an animal, such as a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

As used herein, the terms “comprises”, “comprising”, “containing”, “having”, and the like can mean “includes”, “including”, and the like; “consisting essentially of” or “consists essentially”, likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprise”, “comprising”, and “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Mice Model

Experiments involving mice were performed according to the Hebrew University guidelines and laws, in compliance with the protocols approved by the Hadassah Medical School animal ethics committees. Transgenic mice expressing a tetracycline-regulated trans-activator protein (tTA) mostly in the liver (driver line) were mated with transgenic mice harboring a VEGF164-encoding transgene driven by a tetracycline-responsive promoter (responder line). Pups that inherited both transgenes were selected for modulating VEGF expression, whereas littermates that inherited only the driver transgene served as controls. All mice were kept under tetracycline (160 mg/ml) in sweetened water (2% commercial sugar).

### Levels of Circulating VEGF

Once a month, 100 μl of blood were collected through the tail vein into 10% buffer sodium citrate or K3EDTA. Plasma was prepared by centrifugation (2,060 g for 20 min) and VEGF concentration was measured by Enzyme-linked immunosorbent assay (ELISA) according to manufacturer instructions (R&D Systems).

### Complete Blood Counts (CBC)

Once in three months, 12 μl of blood were collected from the tail vein into 12 μl of heparin solution. CBC was done using an Auto Hematology Analyzer (BC-2800Vet, Mindray).

### Survival Curve

Two cohorts of mice, kept under tetracycline in the drinking water, were monthly weighed and monitored for changes in health status. Mice displaying signs of severe altered health were euthanized according to the Authority for Biological and Biomedical model's policy at the Hebrew University. The survival curve was created using GraphPad prism software.

### Tissue Processing for Histological Analysis

VEGF-treated and control littermates were always sacrificed on the same day. Mice were sacrificed by lethal dose injection of pentobarbital (1 mg/gr mouse). Tissues were harvested, immediately, fixed in 4% buffered formalin and processed for paraffin embedding. Sections of 6 μm were stained routinely with either Hematoxylin and Eosin or Masson trichrome.

### Rotarod Test

Mice were brought to the experimental room 20 min before testing, to ensure they are fully awake. Mice were rested for 20 min by a return to the home cage after each motor test to allow recovery of muscular strength and a return to normal levels of arousal. Mice were placed on the rotating rod, facing away from the direction of rotation so they had to walk forward to stay upright. The start speed was adjusted to 4 rpm and the acceleration rate to 20 rpm/min. Maximum speed was 40 rpm. The time at which the mouse fell was recorded. Each mouse was tested in three trials and mean time was calculated.

### Preparation of Mouse Skeletal Tissue for High-Resolution 3D Confocal Imaging

Bones were collected and immediately fixed in 2% buffered fresh paraformaldehyde for 12 hours. Decalcification was done for 48 hr using a solution of 0.5 M EDTA, pH 7.4 at 4° C. with constant agitation. The bone samples are cryoprotected in a 20% (w/v) sucrose and 2% (w/v) polyvinylpyrrolidone (PVP) before subjecting them to embedding and freezing in a solution of 8% (w/v) gelatin with 20% (w/v) sucrose and 2% (w/v) PVP. For the purposes of 3D imaging, 70-150 μm thick sections were used. For immunohistochemistry, bone sections were permeabilized with a 0.3% (v/v) Triton X-100 solution. To achieve efficient penetration of antibodies into very thick sections (<100 μm), the inventors prepared the primary antibody solution in 0.05% (v/v) Triton X-100 and incubated the sections for 12 hr at 4° C. After extensive washing, the sections were incubated with a fluorescent secondary antibody. Imaging was done using a confocal laser-scanning microscopy, using the z-stack scanning to obtain sequential depth imaging of thick bone sections. Three dimensional (3D) reconstructions of images were done using Imaris software.

### Progression of Kyphosis

Mice were sedated with 50 mg/kg ketamine HCl (Ketamil, Troy Laboratories) in combination with 10 mg/kg xylazine HCl (Ilium Xylazil-20, Troy Laboratories) administered by subcutaneous injection. Mice were lightly taped to the table support. Imaging was done by X-ray radiography (GE OEC 9900 Elite-52 kVp, 4.30 mA). Kyphosis index (KI) was measured as follows:
a) a line was drawn from posterior edge of C7 (A) to the posterior edge of L6 (B), and termed AB line;
b) a line was drawn from the dorsal border of the vertebral body farthest from the AB line, and termed CD line;

$$c ) Kyphosis index ( K I ) = \frac{AB Line}{CD Line}$$

### Example 1

### VEGF Overexpression Increases Life-Span and Prevents Cognitive and Tissue Deterioration

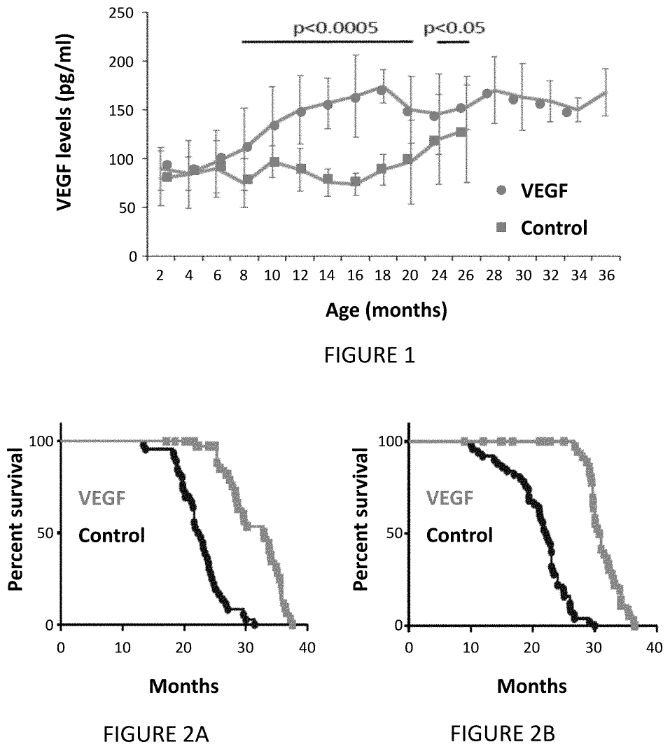

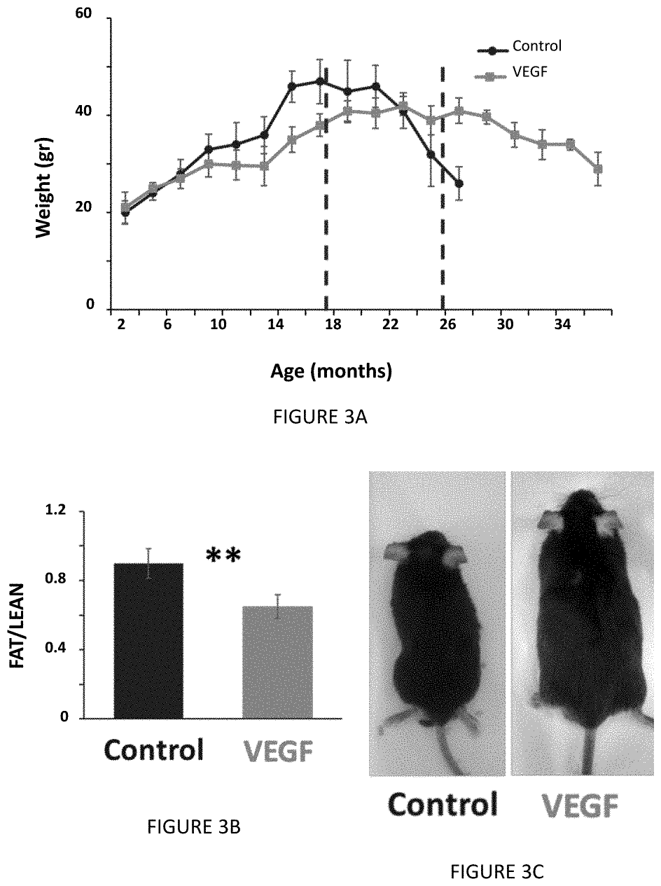

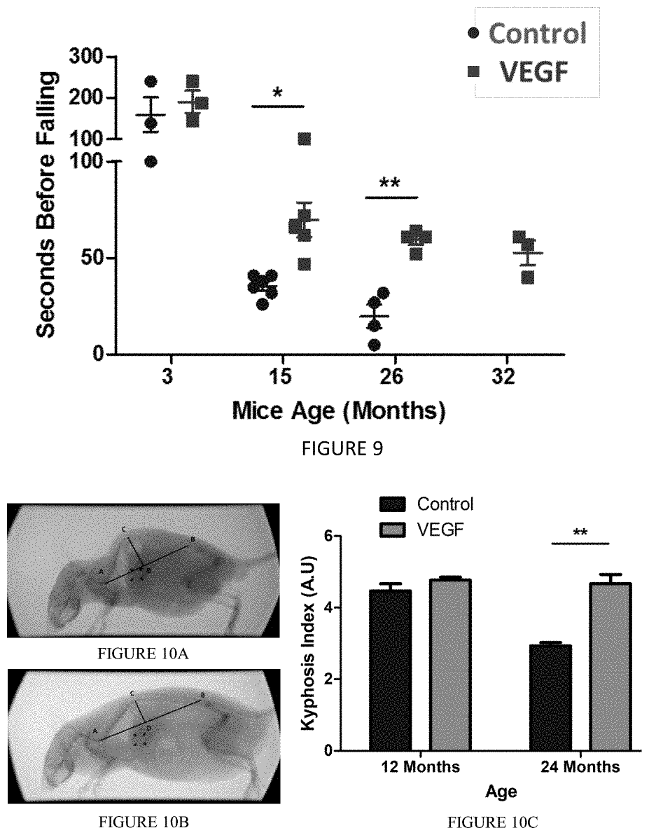

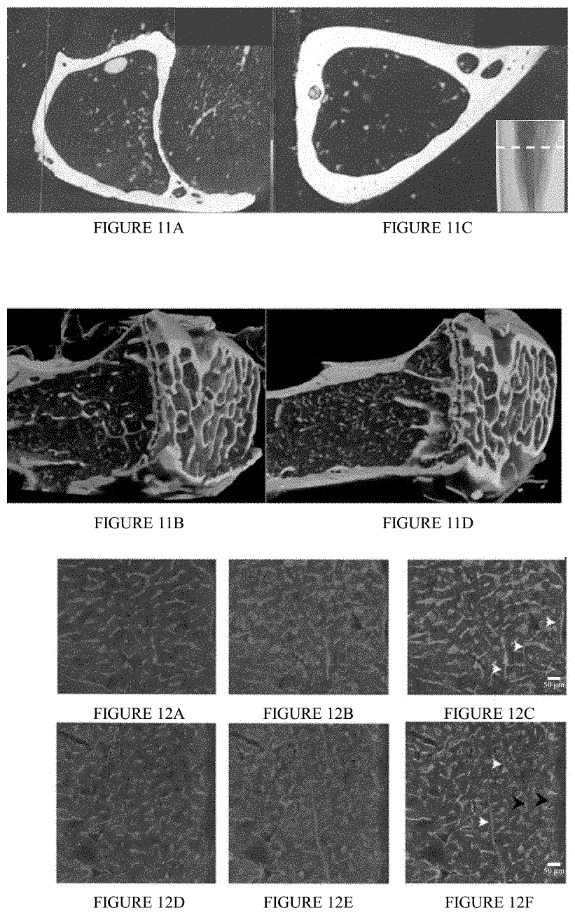

VEGF over-expressing mice were found to have approximately 3-fold at most higher circulating VEGF levels (FIG. 1). The inventors showed that such individuals over-expressing VEGF outlived control littermates by approximately 50% (FIG. 2; median survival 33 months vs 22.7 months for males (2A); 30.85 months vs 22.2 months for females (2B)). Furthermore, the inventors found that during adulthood VEGF-overexpressing mice gained less weight while at the age of 24 months control individuals lost substantially more weight and were leaner than the VEGF over-expressing counterparts, weighing 27±3 gr and 40±5 gr, respectively (FIG. 3). Then, the inventors tested how cognitive performance is affected with respect to VEGF overexpression. In a ROTAROD test model, individuals over-expressing VEGF showed comparable or better performance at any testing event compared to control (FIG. 9). Furthermore, a severer kyphosis was observed in control mice, compared to the VEGF over-expressing mice (FIG. 10). After termination, several tissues were harvested for histological observation. Elongated and branched arteries were observed in the diaphysis of VEGF over-expressing mice, indicating bone perfusion (FIG. 12). Micro computational tomography (CT) images of cross sections through femoral bones showed control mice lost approximately 22% more bone tissue than their VEGF littermates (FIG. 11).

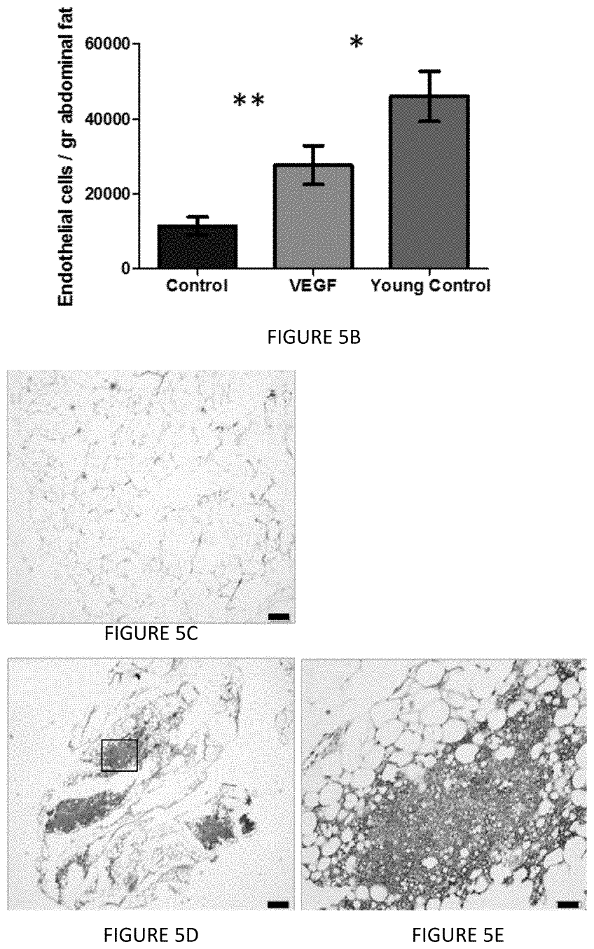

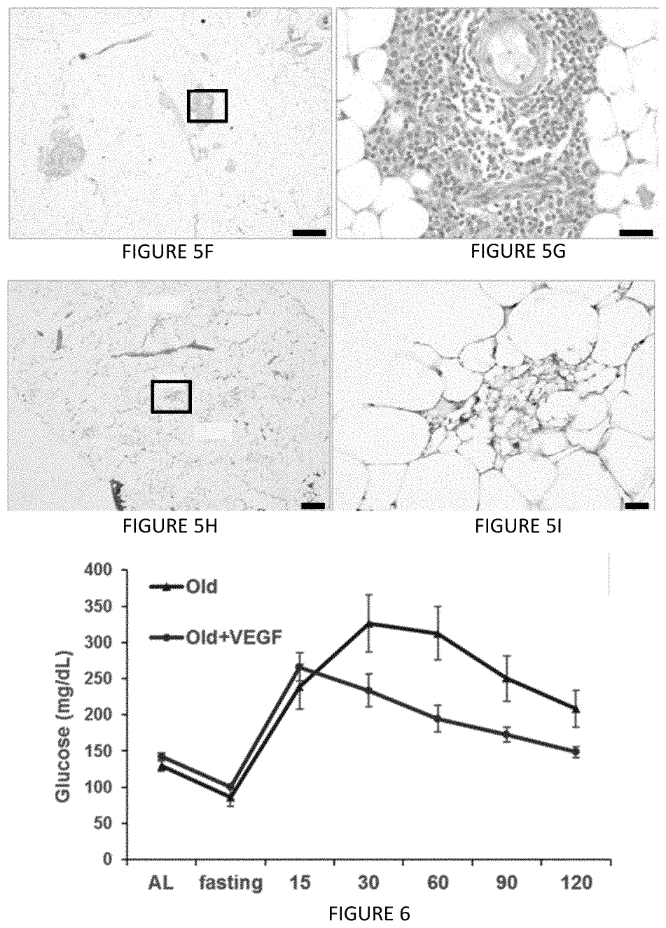

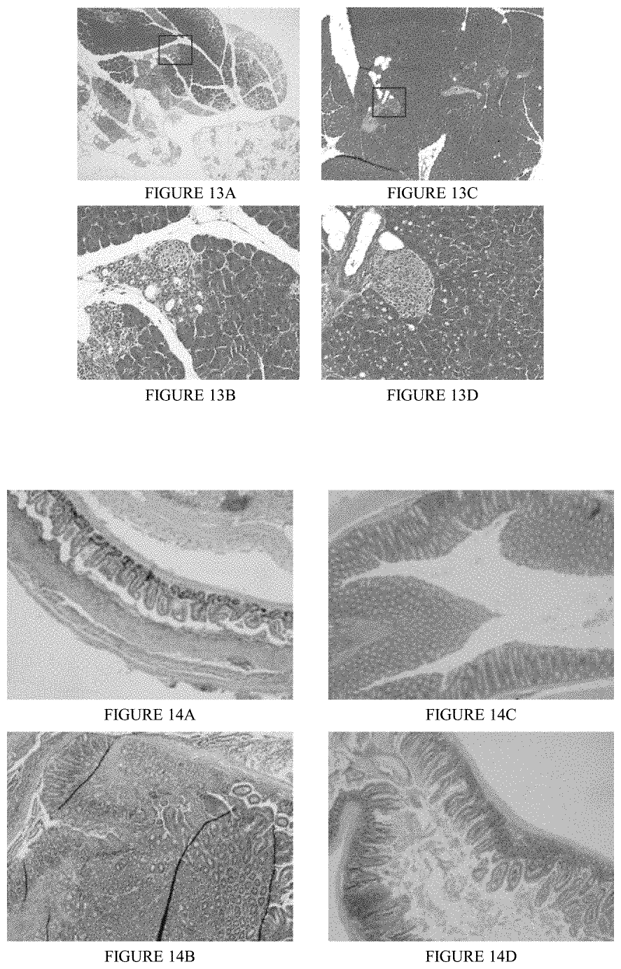

VEGF over-expressing mice were found to have a richer layer of adipose tissue compared to control mice skin, which was found to accommodate a very few adipocytes (FIG. 8). With respect to metabolic tissues, the pancreas, intestine and liver were examined (FIGS. 13,14 and 7, respectively). A significant reduction in white adipose tissue (WAT) mass was observed in VEGF mice compared to their control littermates (FIG. 5A), which was accompanied by adequate perfusion maintenance of this tissue (FIG. 5B). Control WAT was found to accommodate larger immune cell infiltrates (5F-5G), compared to WAT obtained from VEGF (FIGS. 5H-5I). Furthermore, islands of beige adipocytes, which are known to have high thermogenic capacity, were observed only in the VEGF mice (FIG. 5D).

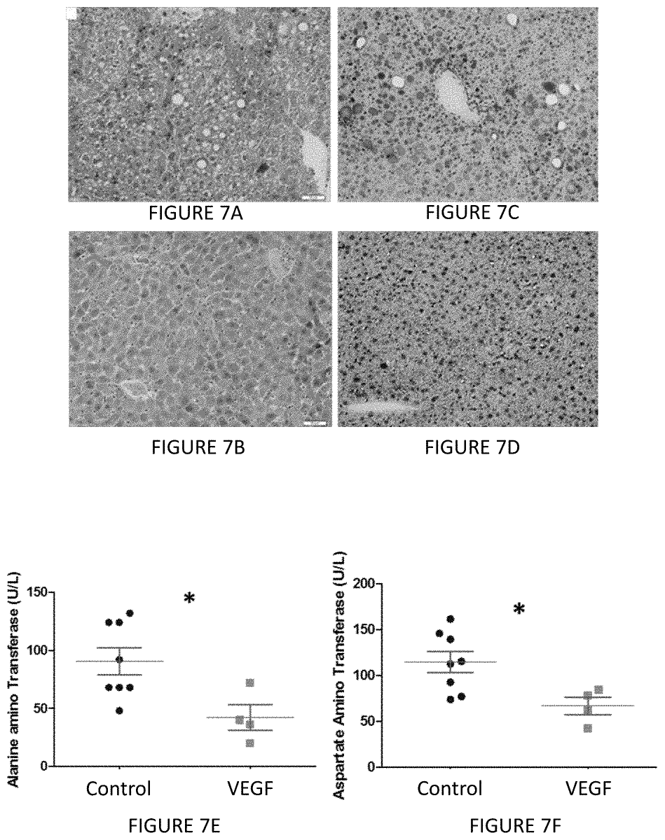

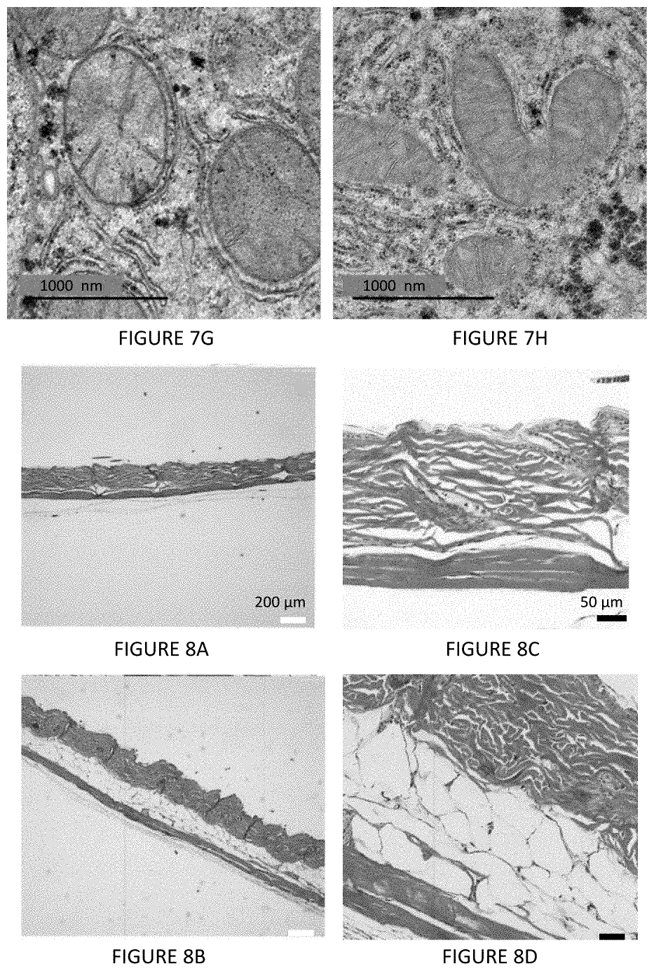

VEGF over-expressing mice demonstrated hallmark features of a healthy pancreas, intestines and liver compared to control, in which different steatosis and adenomas were observed. Hepatic damage was further demonstrated by increased circulating enzymes (FIGS. 7E-7F) and hepatocytic mitochondria rough endoplasmic reticulum morphologies (FIGS. 7G-7H).

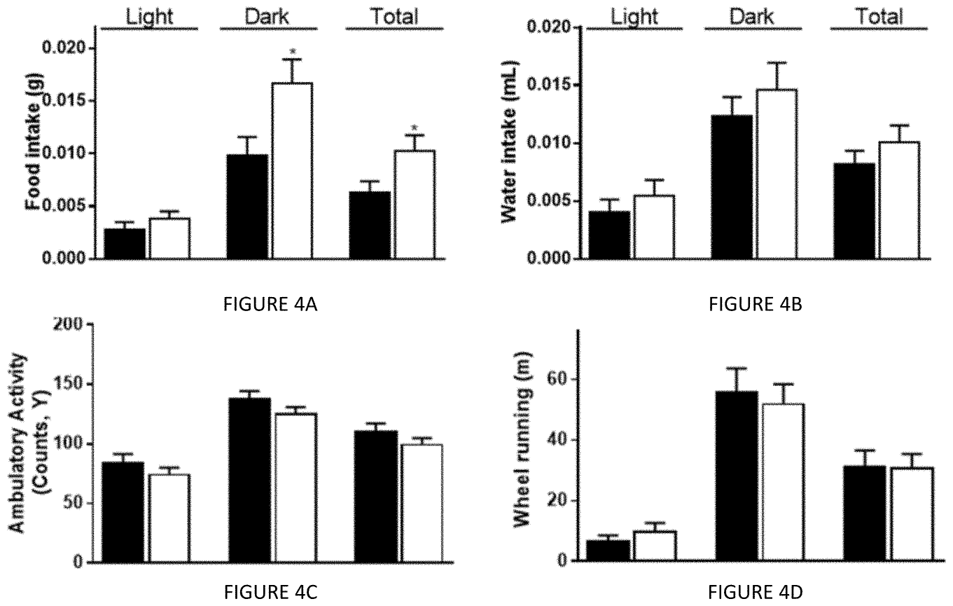

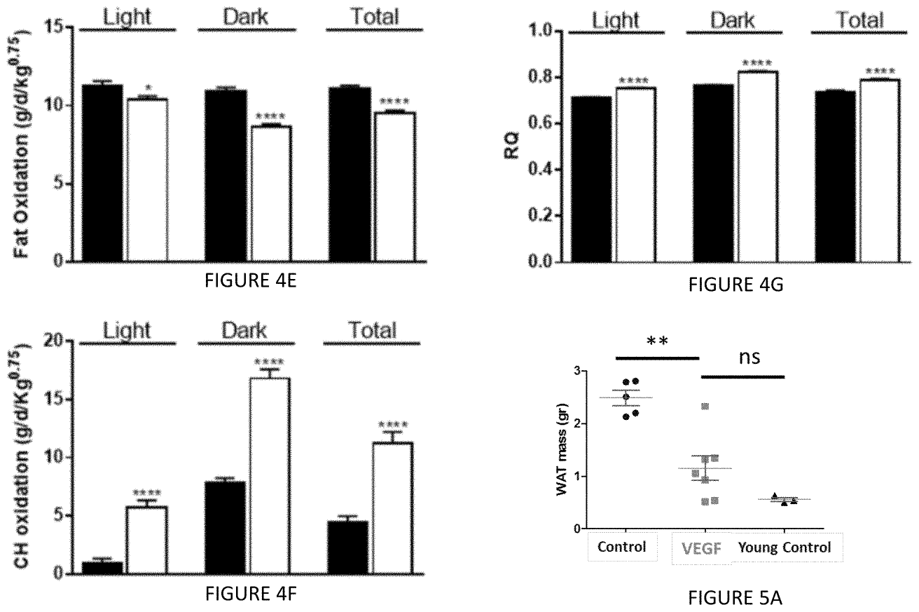

In terms of metabolic activity, VEGF-overexpressing mice were found to have significantly increased food intake (FIG. 4A), conserved a higher metabolic flexibility while aging, and showed better glucose tolerance at the age of 18 months (FIG. 6).

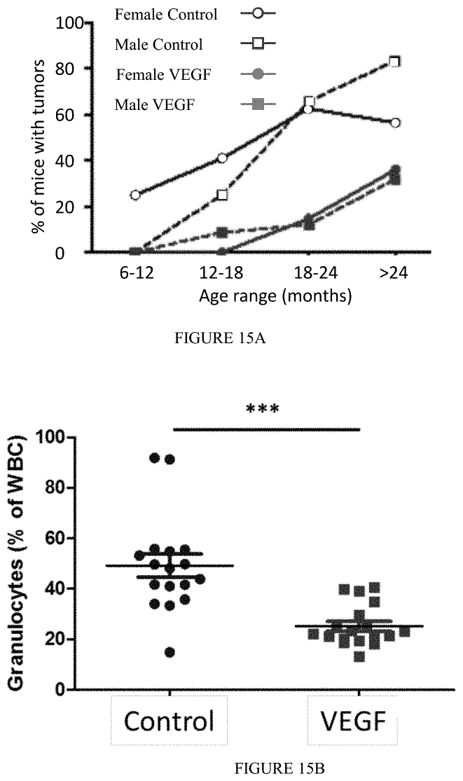

Mice over-expressing VEGF were found to be less prone to spontaneous cancer, as reflected by the percentage of mice presenting at least one spontaneous tumor type at the time of sacrifice. Specifically, in either female or male control mice, neoplastic lesions were observed more often than in the VEGF-overexpressing littermates (FIG. 15A). With this respect, a significant increase in circulating granulocytes was observed in the blood of control mice compared to the VEGF-overexpressing littermates (FIG. 15B).

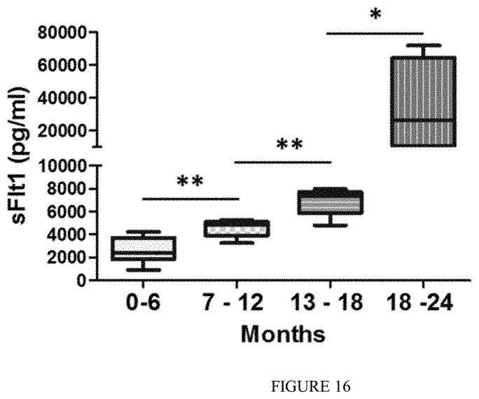

Additionally, the inventors showed that levels of circulating soluble VEGF Receptor 1 (VEGFR1, i.e., sFlt1) the increase in aging control mice (FIG. 16). During the last months of life, control aged mice comprised significantly higher sFlt1levels compared to control young mice. Therefore, increasing the levels of circulating VEGF, by, for example, inhibiting or blocking sVEGFR, can provide a therapeutic effect with respect to age-related disease, disorder, or symptoms thereof.

### Example 2

### Organ Perfusion and Neoplasm Development

Vasculature is visualized using ex-vivo micro-computed tomography (μCT)-based imaging. Anaesthetized mice are injected with μAngiofil® and after polymerization, organs (including but not limited to brain, heart, thymus, lungs, stomach, kidney, liver, ovary or testis, adrenal, skeletal muscle, abdominal fat) are collected and fixed. Samples are scanned using a desktop microCT and blood vessel sizes are assessed (Matlab) and plotted.

Perfused organs can be further inspected for the presence of neoplastic lesions using immunohistochemistry, immunofluorescence, hematoxylin-eosin staining, and others.

As exemplified herein, mice over-expressing VEGF were found to be less prone to spontaneous cancer. This was reflected by the percentage of mice presenting at least one spontaneous tumor type at the time of sacrifice. Specifically, in either female or male control mice, neoplastic lesions were observed more often than in the VEGF-overexpressing littermates (FIG. 15A).

### Example 3

### Plasma Levels of Hormones and Growth Factors

Parathyroid hormone (PTH), Follicular stimulating hormone (FSH), Growth hormone (GH), Insulin-like growth factor 1 (IGF-1), Growth Differentiation Factor 11 (GDF11), Myostatin and Estrogen are quantified by means of ELISA (R&D Systems).

### Example 4

### Bone Density Analysis

Bone parameters were evaluated by bone mineral density using μCT and mechanical testing of the tibia and showed that control mice lost approximately 22% more bone tissue than their VEGF littermates (FIG. 11).

### Example 5

### Cognitive Performance Analysis

Cognitive performance is evaluated by water-maze, fear conditioning assay, open-field and novelty recognition assay, and others.

As exemplified herein, the inventors showed that improved cognitive performance correlated with VEGF overexpression. Specifically, using a ROTAROD test model, the inventors had shown that individuals over-expressing VEGF had comparable or better performance at any testing event compared to control (FIG. 9).

### Example 6

### Fertility

Litter size and number are recorded during 6 months for mice of different ages. Ovaries are collected and processed for immunohistochemistry. Morphological assessment of aging is done according to the number of follicles and corpus luteum as well as atretic follicles.

### Example 7

### Organ Regeneration

Skin wound healing—Two full-thickness excisions that include the panniculus carnosus are created on the dorsum, and a 0.5 mm thick silicone splint is placed around the wound. A translucent occlusive dressing is applied, digital images are taken, and micro-calipers are used to measure the wound area daily. Blood perfusion is determined using laser Doppler perfusion imaging. Ten days after wounding, both wounds are excised for histological and gene analyses.

Liver regeneration after partial hepatectomy—partial hepatectomy is done and regeneration is monitored by MRI, histological measurement of hepatocyte and endothelial cells proliferation.

Hematopoietic recovery after acute radiation—mice are exposed to sublethal dose total body radiation. Hematopoietic recovery is monitored by cell blood counts analysis and measurements of bone marrow and spleen cellularity.

Muscle regeneration—muscle injuries are induced by intramuscular injection of BaCl₂. Regeneration is measured by morphological and morphometric analysis of the regenerating muscle fibers.

### Example 8

### Thymic Atrophy

Thymuses are harvested and weighed. A hemocytometer is used to count the numbers of thymocytes (Trypan blue viability test). Single cell suspension is analyzed by FACS for cellularity and immunophenotyping. Each individual cell data is collected and analyzed using CellQuest.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for preventing or treating an age-related disorder or symptoms thereof in a subject, the method comprising administering to said subject a pharmaceutical composition comprising a therapeutically effective amount of vascular endothelial growth factor (VEGF)-stimulating compound and an acceptable carrier, wherein said composition constantly maintains VEGF plasma levels in said subject by at most 3-fold compared to a baseline, thereby preventing or treating an age-related disorder or symptoms thereof in the subject.

2. The method of claim 1, wherein said VEGF plasma levels comprises free VEGF plasma levels.

3. The method of claim 1, wherein said administering is for at least 30 days before appearance of the age-related disorder or symptom thereof.

4. The method of claim 1, wherein said constantly is for at least 30 days.

5. The method of claim 1, wherein said subject is afflicted with chronic ischemia.

6. The method of claim 5, wherein said subject afflicted with chronic ischemia has plasma lactate levels of 2-5 mmol/L.

7. The method of claim 1, wherein said age-related disorder or symptom is selected from the group consisting of: muscle weakness, cold intolerance, skin wrinkles, reduced skin healing, weight loss, weight gain, cognitive impairment, kyphosis, reduced bone mineralization, inhibition or lack of brown adipose tissue activity, and subdermal fat loss.

8. The method of claim 1, wherein said age-related disorder is selected from the group consisting of: muscle wasting disease, osteoporosis, pancreatic disease, intestinal disease, neoplastic lesions, and hepatic disease.

9. The method of claim 1, wherein said VEGF-stimulating compound is selected from the group consisting of: a nucleic acid, a peptide, a polypeptide, a peptidomimetic, a carbohydrate, a lipid, a small organic molecule, and an inorganic molecule.

10. The method of claim 1, wherein said VEGF-stimulating compound is selected from the group consisting of: VEGF, VEGF Receptor (VEGFR)-stimulating compound, or any combination thereof.

11. The method of claim 1, wherein said baseline is VEGF basal levels in a tissue of said subject.

12. A method for extending the lifespan of a cell, tissue, an organ, or an organism, the method comprising the step of constantly maintaining VEGF levels in said cell, said tissue, said organ, or said organism by at most 3-fold compared to a baseline, thereby extending the lifespan of said cell, said tissue, said organ, or said organism.

13. The method of claim 12, wherein said constantly is for at least 30 days.

14. The method of claim 12, comprising the step of administrating to said cell, said tissue, said organ, or said organism a pharmaceutical composition comprising a therapeutically effective amount of a VEGF-stimulating compound.

15. The method of claim 14, wherein said administering is for at least 30 days.

16. The method of claim 14, wherein said VEGF-stimulating compound is selected from the group consisting of: a nucleic acid, a peptide, a polypeptide, a peptidomimetic, a carbohydrate, a lipid, a small organic molecule and an inorganic molecule.

17. The method of claim 14, wherein said VEGF-stimulating compound is selected from the group consisting of: VEGF, VEGF Receptor (VEGFR)-stimulating compound, or any combination there.

18.-24. (canceled)
